SEQUENCE LISTING (2 sequences)

SEQ ID NO: 1 - protein, 70 aa, synthetic construct
  source 1..70: note = Description ofsequence: PF4 sp|P02776|32-101
EAEEDGDLQCLCVKTTSQVRPRHITSLEVIKAGPHCPTAQLIATLKNGRKICLDLQAPLYKKIIKKLLES

SEQ ID NO: 2 - protein, 28 aa, synthetic construct
  source 1..28: note = fibrinogen gamma-chain C-terminal residues 390-411 with 6xHis tag
HHHHHHNRLTIGEGQQHHLGGAKQAGDV

(19) United States
(12) Patent Application Publication
(10) Pub. No.: US 2025/0003978 A1
(43) Pub. Date: Jan. 2, 2025

(54) DIAGNOSIS AND TREATMENT OF ANTI-PF4 INDUCED THROMBOCYTOPENIA

(71) Applicant: The Regents of the University of California, Oakland, CA (US)

(72) Inventors: Yoshikazu Takada, Oakland, CA (US); Yoko Takada, Oakland, CA (US)

(21) Appl. No.: 18/701,440

(22) PCT Filed: Oct. 11, 2022
(86) PCT No.: PCT/US2022/046249

§ 371 Date: Apr. 15, 2024

Related U.S. Application Data
(60) Provisional application No. 63/371,450, filed on Aug. 15, 2022.
(60) Provisional application No. 63/256,081, filed on Oct. 15, 2021.

(51) Int. Cl. G01N 33/68; C07K 14/745

(52) U.S. Cl. CPC G01N 33/6845; C07K 14/745

(57) ABSTRACT

The present invention resides in the discovery that the presence of anti-PF4 antibody in the PF4-integrin complex promotes platelet aggregation and subsequently thrombocytopenia. PF4 mutants incapable of binding integrin are also disclosed as inhibitors of the PF4-integrin complex and thus useful therapeutic agents against thrombocytopenia. Therefore, this invention provides methods for diagnosis and treatment of thrombocytopenia.

### BACKGROUND OF THE INVENTION

This application claims priority to U.S. Provisional Patent Application No. 63/256,081, filed Oct. 15, 2021, and U.S. Provisional Patent Application No. 63/371,450, filed Aug. 15, 2022, the contents of both are hereby incorporated by reference in the entirety for all purposes.

### BACKGROUND OF THE INVENTION

As the novel SARS-Coronavirus 2 continues to infect a large number of individuals worldwide, one of the leading approaches in handling this global health crisis is vaccination against SARS-CoV2, the causative agent for COVID-19. It has been reported that vaccination may result in, albeit at a low frequency, a vaccine-induced catastrophic thrombotic thrombocytopenia (VITT) disorder. This disorder presents as extensive thrombosis in atypical sites, primarily in the cerebral venous, alongside thrombocytopenia and the production of autoantibody against platelet-factor 4 (PF4, chemokine CXCL4). This adverse effect resembles the clinical presentation of the classical immune-mediated heparin-induced thrombocytopenia (HIT) disorder, which is induced by anti-PF4/heparin complex and occurs following exposure to heparin. VITT is also very similar to autoimmune HIT (aHIT), which is induced by anti-PF4 but none of these patients had been pre-exposed to heparin before disease onset. Anti-PF4 has also been detected in several autoimmune diseases (e.g., SLE, Ulcerative colitis). It is unclear, however, how anti-PF4 induces thrombotic thrombocytopenia.

PF4 is one of the most abundant proteins in platelet granules and rapidly transported to the surface upon platelet activation. A current model of thrombotic thrombocytopenia suggests that (1) anti-PF4 binds to PF4 and induces PF4 clustering, (2) the complex binds to platelets by binding to the FcRγIIA receptor and proteoglycans of platelets, leading to (3) platelet activation and aggregation. PF4 is known to bind to integrins αvβ3 and αMβ2, but the role of integrins in PF4-induced thrombotic thrombocytopenia is unclear. Activation of platelet integrin αIIbβ3 is a key event that leads to αIIbβ3 binding to fibrinogen and platelet aggregation. It was previously shown that chemokines fractalkine and SDF-1 are ligands for several integrins and activate integrins in an allosteric mechanism by binding to the allosteric site (site 2) of these integrins, which is distinct from the classical ligand-binding site (site 1). PF4 is believed to bind to integrin αIIbβ3, a key component of platelet aggregation, and activates it. It was shown that PF4 bound to soluble integrin αIIbβ3 in cell-free conditions and only weakly activated this integrin. Notably, an anti-PF4 antibody (RTO) markedly enhanced PF4-induced activation of soluble αIIbβ3 in heparin-independent manner. PF4 binding to integrin αIIbβ3 may not strongly activate integrin αIIbβ3, but the PF4/anti-PF4 complex markedly strongly activates αIIbβ3 possibly by changing PF4 conformation and results in strong aggregation of platelets. Since RTO does not require heparin, the observed PF4/anti-PF4-induced αIIb3 activation may represent aHIT or VITT, but may or may not for HIT. The goal of this study is to illustrate the mechanism of thrombotic thrombocytopenia by anti-PF4 through allosteric activation of platelet integrin αIIbβ3 by PF4. An activation assay e.g., an ELISA-type assay, can serve as a diagnostic tool of VITT, HIT, and aHIT. Also, compounds that bind PF4 (e.g., nanobodies to PF4) that block PF4-induced αIIbβ3 activation can be used as a therapeutic tool by blocking binding to the allosteric site (site 2). Moreover, PF4 mutants that do not induce αIIbβ3 activation due to presence of mutations in the integrin-binding site in PF4 abolishing PF4-integrin binding are disclosed for their potential utility as therapeutic agents for PF4-induced thrombocytopenia.

Given the prevalence of thrombocytopenia and its significant health implications, there exists an urgent need for developing new compositions and methods effective for the prophylaxis and treatment of the disease. This invention provides information to fulfill these and other related needs.

### BRIEF SUMMARY OF THE INVENTION

The classical immune-mediated heparin-induced thrombocytopenia (HIT) is induced by autoantibody against platelet-factor 4 (PF4)/heparin complex. Vaccine-induced thrombotic thrombocytopenia (VITT) and autoimmune HIT (aHIT) are induced by anti-PF4 in a heparin-independent manner. Activation of platelet integrin αIIbβ3 is a key event that leads to αIIbβ3 binding to fibrinogen and platelet aggregation, but is not involved in current models of HIT or VITT. In this study, PF4 was tested for its binding to site 2 of αIIbβ3 by docking simulation and found to not activate it. On the other hand, PF4/anti-PF4 mAb (RTO, heparin-independent) complex potently activated it at biological concentrations of PF4 (<1 μg/ml), but anti-PF4/heparin (KKO) did not. This indicates that RTO changes the phenotype of PF4 from inhibitory to pro-inflammatory and induces integrin activation. Modified PF4 peptides containing mutations in the predicted site 2 binding interface of PF4 are tested for their effect on integrin αIIbβ3 activation, and a PF4 mutant/RTO complex was found defective in activating integrins. Such PF4 mutants act as antagonists of integrin activation induced by RTO/wild-type PF4. Similar results were obtained with vascular integrin αvβ3. A potential mechanism is therefore proposed, in which RTO/PF4 complex binds to site 2 and activates integrins and triggers thrombocytopenia or other autoimmune diseases. PF4 mutants of this functional profile thus can act as antagonists and serve as therapeutics for autoimmune diseases and conditions including thrombocytopenia.

Accordingly, in a first aspect, the present invention provides an isolated polypeptide acting as an antagonist of wild-type PF4 protein, i.e., capable of disrupting the formation of PF4-integrin (e.g., integrin αIIbβ3 or αvβ3) complex. This isolated polypeptide comprises the amino acid sequence of SEQ ID NO: 1, wherein the amino acid sequence of SEQ ID NO: 1 has at least one (e.g., two or more) mutation at residue(s) R20, R22, K46, R49, K62, K65, and K66, and wherein the polypeptide suppresses formation of the PF4-integrin (e.g., integrin αIIbβ3 or αvβ3) complex, as verified by assay methods known in the pertinent field and/or described herein. In some embodiments, the polypeptide consists of the amino acid sequence of SEQ ID NO: 1, with at least one (e.g., two or more) mutation at residue(s) R20, R22, K46, R49, K62, K65, and K66, and the polypeptide suppresses formation of the PF4-integrin complex. In some embodiments, the amino acid sequence of SEQ ID NO: 1 has R20 and R22 mutated such as by deletion or substitution (e.g., with Glu), for example, the mutation is R20E/R22E double mutant. In some embodiments, the amino acid sequence of SEQ ID NO: 1 has K46 and R49 mutated such as by deletion or substitution (e.g., with Glu), for example, the mutation is K46E/R49E double mutant. In some cases, all four residues R20, R22, K46, and R49 in SEQ ID NO: 1 are mutated such as by deletion or substitution, for example, the mutations are R20E/R22E/K46E/R49E. In some embodiments, the PF4 mutant polypeptide includes, in addition to SEQ ID NO: 1 and mutation(s), at least one possibly two amino acid sequences heterologous to PF4 in origin and located at the N-terminus and/or C-terminus of the polypeptide. In some cases, non-naturally occurring amino acids or amino acid analogs (such as one or more D-amino acids) may be present in a PF4 mutant polypeptide.

This invention resides in the discovery of the role of anti-PF4 antibody in the complex of PF4 with integrin (e.g., integrin αIIbβ3 or αvβ3), thus providing new methods and compositions useful for diagnosing and treating thrombocytopenia induced by anti-PF4 antibody. As such, in a second aspect, the present invention provides a method for diagnosing thrombocytopenia in a patient, who may have shown clinical symptoms indicative of thrombocytopenia. The method involves obtaining a blood sample from the patient and then detecting, in the blood sample, presence of anti-PF4 antibody in a PF4-integrin complex (e.g., PF4 complex with integrin αIIbβ3 or αvβ3). In some embodiments, the thrombocytopenia is a vaccine-induced thrombotic thrombocytopenia (VITT), heparin-induced thrombocytopenia (HIT), autoimmune HIT (aHIT). In some cases, the thrombocytopenia is VITT, and the patient has recently received COVID-19 vaccination, for example, within 24, 48, or 72 hours or within 24-48 or 24-72 hours just prior to the testing. In some cases, the thrombocytopenia is aHIT, and the patient has been diagnosed with COVID-19 and may be actively experiencing COVID symptoms. In some embodiments, an ELISA assay is performed for the detection of anti-PF4 antibody.

The third aspect of the present invention provides a method for preventing or treating thrombocytopenia in a patient in need thereof by administering to the patient an effective amount of a composition comprising an effective amount of a compound inhibiting or disrupting the binding between PF4 and integrin (e.g., integrin αIIbβ3 or αvβ3). In some embodiments, the thrombocytopenia is a vaccine-induced thrombotic thrombocytopenia (VITT), heparin-induced thrombocytopenia (HIT), autoimmune HIT (aHIT). In some cases, the thrombocytopenia is VITT, and the patient has received COVID-19 vaccination within the past 24-48 or 24-72 hours. In some cases, the thrombocytopenia is aHIT, and the patient has been diagnosed with COVID-19 and optionally is experiencing one or more COVID symptoms. In some embodiments, the inhibitor is a compound that interferes with binding between PF4 and integrin (e.g., integrin αIIbβ3 or αvβ3) and thus inhibits complex formation between the two molecules. In some embodiments, the inhibitor is an antibody against PF4 that interferes with and thus inhibits binding between PF4 and integrin (e.g., integrin αIIbβ3 or αvβ3), such as a single chain antibody (ScFv) or a nanobody for PF4. In some embodiments, the inhibitor is a PF4 mutant with reduced binding to integrin (e.g., integrin αIIbβ3 or αvβ3), for example, a PF4 mutant that contains one or more mutations within a region of the PF4 protein normally involved in interacting with integrin (e.g., integrin αIIbβ3 or αvβ3), thus causing the mutant to reduce or lose PF4's original ability to bind integrin (e.g., integrin αIIbβ3 or αvβ3) and form a PF4-integrin complex. For example, the PF4 mutant contains at least one (e.g., two or more) mutation at residue(s) R20, R22, K46, R49, K62, K65, and K66 of SEQ ID NO: 1, including but not limited to, a double mutant R20E/R22E, a double mutant K46E/R49E, a quadruple mutant R20E/R22E/K46E/R49E as described herein.

In a fourth aspect, the present invention provides a screening method for identifying an inhibitor of PF4-integrin (e.g., integrin αIIbβ3 or αvβ3) binding. The method includes these steps: (1) contacting an integrin (e.g., integrin αIIbβ3 or αvβ3) and a polypeptide comprising the amino acid sequence of PF4 protein (e.g., SEQ ID NO:1), in the presence of a test compound, under conditions permissible for PF4-integrin binding; and (2) detecting the level of polypeptide-integrin binding. If a decrease in the level of binding is detected when compared with the level of binding in the absence of the test compound, the decrease indicates the compound as an inhibitor of PF4-integrin binding. Conversely, if an increase in the level of binding is detected when compared with the level of binding in the absence of the test compound, the compound is indicated as an enhancer or promoter of PF4-integrin (e.g., integrin αIIbβ3 or αvβ3) binding. In some embodiments, the integrin is expressed on a cell surface. In some embodiments, the screening method may be carried out in a cell-free experimental system where protein-protein interaction is measured in vitro.

In a further aspect of this invention, a kit is provided for inhibiting thrombosis. Typically, the kit includes a plurality of containers, with a first container containing an inhibitor of binding between PF4 and integrin (e.g., integrin αIIbβ3 or αvβ3) and a second container containing another, known anti-thrombosis therapeutic agent. In some embodiments, the inhibitor is an antibody against PF4 that interferes with and therefore inhibits binding between PF4 and integrin, such as a single chain antibody (ScFv) or a nanobody for PF4. In some embodiments, the inhibitor is a PF4 mutant with reduced binding to integrin, for example, a PF4 mutant that contains one or more mutations within a region of the PF4 protein normally involved in interacting with integrin (e.g., integrin αIIbβ3 or αvβ3), thus causing the mutant to reduce or lose PF4's original ability to bind integrin and form a PF4-integrin complex. For example, the PF4 mutant contains at least one (e.g., two or more) mutation at residue(s) R20, R22, K46, R49, K62, K65, and K66 of SEQ ID NO: 1, including but not limited to, a double mutant R20E/R22E, a double mutant K46E/R49E, a quadruple mutant R20E/R22E/K46E/R49E as described herein.

### BRIEF DESCRIPTION OF THE DRAWINGS

FIG. 1. Docking models of anti-PF4/PF4-integrin interaction. FIG. 1a PF4 binding to the classical ligand-binding site (site 1) of active αvβ3 (1L5G.pdb). 3D structure of αvβ3 was used because active and inactive 3D structures are known. Autodock3 was used for docking simulation. The simulation predicts that PF4 binds to site 1 (docking energy—23.4 kcal/mol). FIG. 1b PF4 binding to the allosteric site (site 2) of inactive αvβ3 (1JV2.pdb). Docking energy-21.2 kcal/mol. Docking models in FIG. 1a and FIG. 1b were superposed (FIG. 1c). FIG. 1d When anti-PF4 (RTO)/PF4 complex structure (4RAU.pdb) was superposed, PF4/anti-PF4 is predicted to bind to αvβ3 (site 2) without steric hindrance. It is hypothesized that PF4 binds to the site 2 of inactive integrins but does not induce activation at biological concentrations of PF4 (FIGS. 3 and 4). Mutations were introduced in the predicted site 2-binding interface of PF4. Positions of amino acid residues selected for mutagenesis (Arg20, Arg22, Lys46, and Arg49) are shown. FIG. 1e The anti-PF4/PF4 complex induces integrin activation although PF4 does not. Anti-PF4 is detected in thrombocytopenia and other autoimmune diseases, and this activation by anti-PF4/PF4 may be potentially involved in the pathogenesis of diseases. This model also predicts that PF4 mutants defective in binding to site 3 may be defective in inducing integrin activation and potentially act as antagonists.

FIG. 2. PF4 specifically binds to soluble αIIbβ3 and αvβ3 in ELISA-type binding assays in 1 mM Mn²⁺. FIG. 2a PF4 binds to soluble integrins in 1 mM Mn²⁺ in ELISA-type binding assays. PF4 was immobilized to wells of 96-well microtiter plate and incubated with soluble αIIbβ3 or αvβ3 (1 μg/ml) in Tyrode-HEPES buffer with 1 mM Mn²⁺ (to activate integrins) for 1 hr at room temperature and bound integrins were quantified using anti-β3 (mAb AV10) and HRP-conjugated anti-mouse IgG. Data are shown as means+/−SD in triplicate experiments. The data show that PF4 binds to these integrins at Kd<1 μg/ml. FIG. 2b Binding of authentic PF4 (Invitrogen) to integrins. Binding assays were performed as described in (a). PF4 (6.25 μg/ml) was used. Data are shown as means+/−SD in triplicate experiments. The data show that PF4 binding to soluble integrins is not due to the source of PF4. FIGS. 2c and d The binding of PF4 to integrins was suppressed by the distintegrin domain of ADAM15 fused to GST (ADAM15 disintegrin), but not by control GST. To establish the specificity of PF4 binding to soluble integrins αIIbβ3 (b) and αvβ3 (c), if ADAM15 disintegrin, which is known to bind to integrins αIIbβ3 (22) and αvβ3 (21), suppress the binding. ADAM15 disintegrin (100 μg/ml) suppressed the integrin binding to immobilized PF4 (12.5 g/ml), but control GST (100 μg/ml) did not. Data are shown as means+/−SD in triplicate experiments. This indicates that the binding of soluble integrins to PF4 is specific. FIGS. 2e and f Effect of antagonists to integrins on PF4 binding. Wells of 96 well microtiter plates were coated with PF4 (50 μg/ml) and incubated with soluble integrins (1 μg/ml) in Tyrode-HEPES buffer with 1 mM Mn²⁺ in the presence of mAb 7E3 (specific to human β3, 10 μg/ml), eptifibatide (specific to αIIbβ3, 0.65 μg/ml), or cyclic RGDfV (specific to αvβ3, 10 μM). Assays were performed as described in (a). Data is shown as means+/−SD in triplicate experiments. FIGS. 2g and h Cation dependency of PF4 binding to integrins. Wells of 96-well microtiter plates were coated with PF4 (50 μg/ml) or full-length γC (50 μg/ml), which binds to αIIbβ3 and αvβ3, as a positive control. Wells were incubated with soluble integrins (1 μg/ml) in Tyrode-HEPES buffer with different cations (1 mM). Bound integrins were quantified as described in (a). Data are shown as means+/−SD in triplicate experiments.

FIG. 3. Anti-PF4/PF4 complex potently activated integrin αIIbβ3, but PF4 itself did not. FIG. 3a. Anti-PF4 (RTO) markedly enhances PF4-induced activation of soluble αIIbβ3 in 1 mM Ca²⁺ in ELISA-type activation assays in a dose-dependent manner at physiological concentrations of PF4 (below 1 μg/ml). The fibrinogen fragments γC390-411 (a specific ligand for αIIbβ3) was immobilized to wells of 96-well microtiter plate. Wells were incubated with soluble αIIbβ3 (1 μg/ml) in Tyrode-HEPES buffer with PF4 in 1 mM Ca²⁺ (to keep integrins inactive) for 1 hr at room temperature. Anti-PF4 (RTO or KKO, 10 μg/ml) were added (without heparin). After washing, bound integrins were quantified using anti-β3 (mAb AV10) and HRP-conjugated anti-mouse IgG. After washing, bound integrins were quantified using anti-β3 (mAb AV10) and HRP-conjugated anti-mouse IgG. Mouse IgG (10 μg/ml) and no antibody were used as negative controls. Data are shown as means+/−SD in triplicate experiments. FIG. 3b. Authentic PF4/anti-PF4 (RTO) complex markedly activated integrin αIIbβ3 at 1 μg/ml PF4, but authentic PF4/KKO complex did not. Commercial PF4 was used instead of our own PF4 preparation. Activation assays were performed as described in a. The data show that RTO/authentic PF4 complex activated αIIbβ3 at 1 μg/ml, but the authentic PF4/KKO complex, PF4 with mouse IgG, PF4 itself, did not. FIG. 3c. Point mutations in the predicted integrin-binding site (site 2) of PF4 suppressed activation of αIIbβ3 by the anti-PF4/PF4 complex. The PF4 mutation (R20E/R22E/K46E/R49E) most effectively suppressed integrin activation by anti-PF4/PF4 complex. Positions of the amino acids are shown in FIG. 1d. FIG. 3d. The R20E/R22E/K46E/R49E mutant suppressed integrin activation by the anti-PF4/PF4 complex. WT PF4 (0.5 g/ml) and excess PF4 mutant (5 or 10 μg/ml) were used. The data indicate that the R20E/R22E/K46E/R49E mutant acted as an antagonist.

FIG. 4. Anti-PF4/PF4 complex potently activated integrin αvβ3, but PF4 itself did not. FIG. 4a. Anti-PF4 (RTO) markedly enhances PF4-induced activation of soluble αvβ3 in 1 mM Ca²⁺ in ELISA-type activation assays. Fibrinogen fragment γC399tr (151-399 segment of fibrinogen γ chain, a specific ligand for αvβ3) was immobilized to wells of 96-well microtiter plate. Wells were incubated with soluble αvβ3 (1 μg/ml) in Tyrode-HEPES buffer with PF4 in 1 mM Ca²⁺ (to keep integrins inactive) for 1 hr at room temperature. Anti-PF4 (RTO or KKO, 10 μg/ml) were added (without heparin). After washing, bound integrins were quantified using anti-β3 (mAb AV10) and HRP-conjugated anti-mouse IgG. After washing, bound integrins were quantified using anti-β3 (mAb AV10) and HRP-conjugated anti-mouse IgG. Mouse IgG (10 μg/ml) and no antibody were used as negative controls. (n=3) The data show that PF4 itself did not activate αvβ3 at <1 μg/ml, but the RTO/PF4 complex did. FIG. 4b. Authentic PF4/anti-PF4 (RTO) complex markedly activated integrin αvβ3 at 1 μg/ml PF4, but authentic PF4/KKO complex did not. Commercial PF4 was used instead of our own PF4 preparation. Activation assays were performed as described in a. The data show that RTO/authentic PF4 complex activated αvβ3 at 1 g/ml, but the authentic PF4/KKO complex, PF4 with mouse IgG, PF4 itself, did not. FIG. 4c. Point mutations in the predicted integrin-binding site (site 2) of PF4 suppressed activation of αvβ3 by the anti-PF4/PF4 complex. The PF4 mutation (R20E/R22E/K46E/R49E) most effectively suppressed integrin activation by anti-PF4/PF4 complex. FIG. 4d. The R20E/R22E/K46E/R49E mutant suppressed integrin activation by the RTO/PF4 complex. WT PF4 (0.5 μg/ml) and excess PF4 mutant (5 or 10 μg/ml) were used. The data indicate that the R20E/R22E/K46E/R49E mutant acted as an antagonist.

### DEFINITIONS

As used herein, “PF4” refers to Platelet Factor 4, a small cytokine belonging to the CXC chemokine family and also known as chemokine (C—X—C motif) ligand 4 (CXCL4). This 70-amino acid protein chemokine is released from alpha-granules of activated platelets during platelet aggregation, binds with high affinity to heparin, and promotes blood coagulation by moderating the effects of heparin-like molecules. In this application, the term “PF4” encompasses any naturally occurring human PF4 protein (exemplified herein as SEQ ID NO: 1), its polymorphic variants and species orthologs or homologs. A “PF4 polynucleotide” refers to a nucleic acid sequence from the gene encoding the PF4 protein and may include both the coding and non-coding regions. “PF4 cDNA,” “PF4 mRNA,” “PF4 coding sequence,” and their variations refer to a nucleic acid sequence that encodes a PF4 polypeptide. An exemplary human PF4 amino acid sequence is provided as SEQ ID NO: 1 (EAEEDGDLQCLCVKTTSQVRPRHITSLEVIKAGPHCPTAQLIATLKNGRKICLDLQAP LYKKIIKKLLES).

“A PF4 dominant negative mutant” or “a PF4 mutant” as used herein refers to an PF4 antagonist compound in the form of a mutated PF4 or a fragment thereof, which suppresses anti-PF4/PF4 complex-induced cellular signaling by way of its interaction with integrins (such as integrin αIIbβ3) in a manner that imposes an inhibitory or disruptive effect on the specific binding among anti-PF4/wild-type PF4 and integrin, thus inhibiting downstream events normally triggered by anti-PF4/PF4 signaling, for example, anti-PF4/PF4 complex-mediated blood clotting (thrombosis), inflammatory responses, and autoimmune reactions. In an exemplary PF4 dominant negative mutant, one or more amino acid residues predicted to interact with integrin (especially integrin αIIbβ3), e.g., Arg-20, Arg-22, Lys-46, Arg-49, Lys-62, Lys-65, and Lys-66 residues, are mutated, either by deletion or by substitution with a different amino acid (e.g., Glu), resulting in the mutant having decreased or even abolished capability to bind integrin such as αIIbβ3. These PF4 dominant negative mutants can be identified based on their deficiency compared to the wild-type PF4 in integrin binding, as well as in signaling functions (failure to activate thrombosis for example) in test cells (e.g., endothelial cells). They can also be identified by their capability to suppress anti-PF4/PF4 signaling induced by wild-type PF4 in test cells such as endothelial cells, in addition to their anti-inflammatory activity. A PF4 dominant negative mutant may be initially generated based on the wild-type PF4 amino acid sequence (i.e., SEQ ID NO: 1) with certain amino acid residue(s) mutated, it may further include one or more heterologous amino acid sequences (derived from a source other than the wild-type PF4 protein) at its N-terminus and/or C-terminus. For example, a PF4 dominant negative mutant may optionally include one or more additional heterologous amino acid sequence(s) of 1, 2, 3, 4, 5, 6, 7, 8, 9, 10, 11, 12, 13, 14, 15, 16, 17, 18, 19, 20, 25, 30, 35, 40, 45, or up to 50 amino acids at C- and/or N-terminus of the PF4 sequence. Such heterologous peptide sequences can be of a varying nature, for example, any one of the “tags” known and used in the field of recombinant proteins: a peptide tag such as an AviTag, a peptide allowing biotinylation by the enzyme BirA and so the protein can be isolated by streptavidin, a Calmodulin-tag, a peptide bound by the protein calmodulin, a polyglutamate tag, a peptide binding efficiently to anion-exchange resin such as Mono-Q, an E-tag, a peptide recognized by an antibody, a FLAG-tag, a peptide recognized by an antibody, an HA-tag, a peptide recognized by an antibody, a His-tag, 5-10 histidines bound by a nickel or cobalt chelate, a Myc-tag, a short peptide recognized by an antibody, an S-tag, an SBP-tag, a peptide that specifically binds to streptavidin, a Softag 1 for mammalian expression, a Softag 3 for prokaryotic expression, a Strep-tag, a peptide that binds to streptavidin or the modified streptavidin called streptactin (Strep-tag II), a TC tag, a tetracysteine tag that is recognized by FLASH and ReAsH biarsenical compounds, a V5 tag, a peptide recognized by an antibody, a VSV-tag, a peptide recognized by an antibody, an Xpress tag; or a covalent peptide tags such as an Isopeptag, a peptide that binds covalently to pilin-C protein, a SpyTag, a peptide that binds covalently to SpyCatcher protein; or a protein tag such as a BCCP tag (Biotin Carboxyl Carrier Protein), a protein domain biotinylated by BirA enabling recognition by streptavidin, a Glutathione-S-transferase (GST) tag, a protein that binds to immobilized glutathione, a Green fluorescent protein (GFP) tag, a protein that is spontaneously fluorescent and can be bound by nanobodies, a Maltose binding protein (MBP) tag, a protein that binds to amylose agarose, a Nus-tag, a Thioredoxin-tag, an Fc-tag, derived from immunoglobulin Fc domain, allow dimerization and solubilization. A tag that can be used for purification on Protein-A Sepharose; as well as other types of tags such as the Ty tag. Furthermore, the PF4 dominant negative mutants may also include one or more D-amino acids or include chemical modifications such as glycosylation, PEGylation, crosslinking, and the like.

“Inflammation” is a refers to an organism's immune response to irritation, toxic substances, pathogens, or other stimuli. The response can involve innate immune components and/or adaptive immunity. Inflammation is generally characterized as either chronic or acute. Acute inflammation is characterized by redness, pain, heat, swelling, and/or loss of function due to infiltration of plasma proteins and leukocytes to the affected area. Chronic inflammation is characterized by persistent inflammation, tissue destruction, and attempts at repair. Monocytes, macrophages, plasma B cells, and other lymphocytes are recruited to the affected area, and angiogenesis and fibrosis occur, often leading to scar tissue.

An “inflammatory condition” is one characterized by or involving an inflammatory response, as described above. A list of exemplary inflammatory conditions includes: asthma, autoimmune disease, chronic inflammation, chronic prostatitis, glomerulonephritis, hypersensitivities and allergies, skin disorders such as eczema, inflammatory bowel disease, pelvic inflammatory disease, reperfusion injury, rheumatoid arthritis, transplant rejection, and vasculitis.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H} I by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see. e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., single chain Fv, a chimeric or humanized antibody). One example is the so-called “nanobody” or single-domain antibody (sdAb), an antibody fragment consisting of a single monomeric variable antibody domain, especially a heavy chain variable domain. Like a whole antibody, a nanobody is able to bind selectively to a specific antigen. With a molecular weight of only 12-15 kDa, nanobodies are much smaller than common antibodies (150-160 kDa) having two heavy chains and two light chains, and even smaller than Fab fragments (˜50 kDa) and single-chain variable fragments (˜25 kDa).

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “heterologous,” as used in the context of describing the relative location of two elements, refers to the two elements such as two polynucleotide sequences (e.g., a promoter and a polypeptide-encoding sequence) or polypeptide sequences (e.g., a first amino acid sequence (such as one set forth in SEQ ID NO: 1 with mutation or mutations) and a second peptide sequence serving as a fusion partner with the first amino acid sequence) that are not naturally found in the same relative position. Thus, a “heterologous promoter” of a gene refers to a promoter that is not naturally operably linked to that gene. Similarly, a “heterologous polypeptide/amino acid sequence” or “heterologous polynucleotide” to an amino acid sequence or its encoding sequence is one derived from a non-PF4 origin or derived from PF4 but not naturally connected to the first PF4-derived sequence (e.g., one set forth in SEQ ID NO: 1) in the same fashion. The fusion of a PF4-derived amino acid sequence (or its coding sequence) with a heterologous polypeptide (or polynucleotide sequence) does not result in a longer polypeptide or polynucleotide sequence that can be found naturally in PF4.

The term “inhibiting” or “inhibition,” as used herein, refers to any detectable negative effect on a target biological process, such as RNA/protein expression of a target gene, the biological activity of a target protein, protein-protein specific binding or interaction, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., PF4 and integrin binding), or any one of the downstream parameters mentioned above, when compared to a control. “Inhibition” further includes a 100% reduction, i.e., a complete elimination, prevention, or abolition of a target biological process or signal or disease/symptom. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal or disease/symptom. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or symptom/disease incidence.

As used in this application, an “increase” or a “decrease” refers to a detectable positive or negative change in quantity from a comparison control, e.g., an established standard control (such as an average level of PF4 binding to integrin αIIbβ3). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

A composition “consisting essentially of a PF4 dominant negative mutant” is one that includes a PF4 mutant that inhibits specific binding between wild-type PF4 and integrin (such as integrin αIIbβ3) but no other compounds that contribute significantly to the inhibition of the binding. Such compounds may include inactive excipients, e.g., for formulation or stability of a pharmaceutical composition, or active ingredients that do not significantly contribute to the inhibition of PF4-integrin binding. Exemplary compositions consisting essentially of a PF4 dominant negative mutant include therapeutics, medicaments, and pharmaceutical compositions.

As used herein, an “effective amount” or a “therapeutically effective amount” means the amount of a compound that, when administered to a subject or patient for treating a disorder, is sufficient to prevent, reduce the frequency of, or alleviate the symptoms of the disorder. The effective amount will vary depending on a variety of the factors, such as a particular compound used, the disease and its severity, the age, weight, and other factors of the subject to be treated. Amelioration of a symptom of a particular condition by administration of a pharmaceutical composition described herein refers to any lessening, whether permanent or temporary, that can be associated with the administration of the pharmaceutical composition. For example, the amount of a PF4 dominant negative mutant is considered therapeutically effective for treating a condition involving undesired thrombosis or other inflammatory responses when treatment results in eliminated symptoms, delayed onset of symptoms, or reduced frequency or severity of symptoms such as blood clotting, autoimmune responses, etc.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “subject,” or “subject in need of treatment,” as used herein, refers to an individual who seeks medical attention due to risk of, or actual sufference from, a condition involving an undesirable or abnormal thrombotic process or inflammatory response. The term subject can include both animals, especially mammals, and humans. Subjects or individuals in need of treatment include those that demonstrate symptoms of undesirable or inappropriate thrombosis such as thrombocytopenia and autoimmune response or are at risk of later developing these conditions and/or related symptoms.

### DETAILED DESCRIPTION OF THE INVENTION

### I. Introduction

The vaccine-induced catastrophic thrombotic thrombocytopenia (VITT) disorder presents as extensive thrombosis, thrombocytopenia and the production of autoantibody against platelet-factor 4 (PF4, CXCL4). This resembles the clinical presentation of the classical immune-mediated heparin-induced thrombocytopenia (HIT) disorder, which is induced by anti-PF4/heparin complex and occurs following exposure to heparin. VITT is also very similar to autoimmune HIT (aHIT), which is induced by anti-PF4 but none of these patients had been pre-exposed to heparin before disease onset. Anti-PF4 has also been detected in several autoimmune diseases (e.g., SLE). It is unclear, however, how anti-PF4 is involved in the pathogenesis of these diseases. PF4 is one of the most abundant proteins in platelet granules and rapidly transported to the surface upon platelet activation. Activation of platelet integrin αIIbβ3 is a key event that leads to αIIbβ3 binding to fibrinogen and platelet aggregation. Current models of thrombotic thrombocytopenia (TT) does not include activation of αIIbβ3. It was previously shown that chemokines fractalkine and SDF-1 activate integrins in an allosteric mechanism by binding to the allosteric site (site 2) of these integrins, which is distinct from the classical ligand-binding site (site 1). It is speculated that PF4 binds to integrin αIIbβ3 and activates it. It now has been shown that PF4 binds to soluble integrin αIIbβ3 in cell-free conditions but does not activate this integrin at physiological PF4 concentrations (<1 μg/ml).

Notably, an anti-PF4 (RTO)/PF4 complex potently activated soluble αIIbβ3 in heparin-independent manner. It is believed that anti-PF4 changes the conformation of PF4 and strongly activates αIIbβ3 by binding to site 2, which then results in strong aggregation of platelets. Since the anti-PF4 antibody RTO does not require heparin, the PF4/anti-PF4-induced αIIbβ3 activation may represent aHIT or VITT, but not HIT. These studies connect anti-PF4, PF4, and activation of αIIbβ3, and the subsequent platelet aggregation, and thereby fill in a huge gap in knowledge. It has also been shown that PF4/anti-PF4 potently activates vascular integrin αvβ3, which may play a critical role in autoimmune diseases. The goal of this study is to illustrate the role of anti-PF4/PF4 in thrombosis and autoimmune diseases through allosteric activation of platelet integrins. The present inventors have developed a PF4 mutant that does not induce αIIbβ3 and αvβ3 activation by mutating the site 2-binding site in PF4. The PF4 mutant acted as an antagonist of anti-PF4/PF4-induced activation of integrins in ELISA-type activation assays, indicating it has potential as a therapeutic. Nanobodies to PF4 will be developed and screened for those that block PF4-induced αIIbβ3 activation by blocking binding to the allosteric site (site 2). Such nanobodies possess strong therapeutic potential. The ELISA-type activation assay can serve as a potential diagnostic tool of VITT, aHIT and other autoimmune diseases. To achieve these goals, two studies are performed: 1. study activation of αIIbβ3 induced by anti-PF4/PF4 complex and thromboembolism. First, the PF4 mutant defective in site 2 binding and activation is used to study the mechanism of integrin activation by PF4. Second, the inhibitory PF4 mutant is studied in anti-PF4/PF4-induced integrin activation and cell aggregation in CHO cells that express recombinant αIIbβ3 and in platelets. Third, experiments are performed to reveal whether anti-PF4 agents in patients with autoimmune diseases activate integrins and to establish the role of anti-PF4 in the pathogenesis of diseases as well as the therapeutic potential of PF4 mutants with inhibitory effects on the formation of PF4-integrin αIIbβ3 complex. 2. Screen nanobodies that bind to PF4 and block PF4-induced integrin activation. To study the potential mechanism of integrin activation by anti-PF4 in aHIT, VITT or autoimmune diseases, nanobodies are screened for those that bind to PF4 and activate integrins or block PF4-induced activation. Such antibodies can facilitate the study of PF4/anti-PF4-induced integrin activation. This study illustrates that the binding of anti-PF4 to PF4 enhances PF4-mediated integrin activation, leading to platelet aggregation and thrombosis or activation of many cell types (e.g., monocytes) leading to systemic inflammatory responses.

### II. Recombinant Expression of Polypeptides

### A. General Recombinant Technology

Basic texts disclosing general methods and techniques in the field of recombinant genetics include Sambrook and Russell, Molecular Cloning. A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The sequence of a human PF4 gene, a polynucleotide encoding a polypeptide having the amino acid sequence SEQ ID NO: 1 or its variants/mutants, and synthetic oligonucleotides can be verified after cloning or subcloning using, e.g., the chain termination method for sequencing double-stranded templates of Wallace et al., Gene 16:21-26 (1981).

### B. Chemical Synthesis of Peptides

The amino acid sequence of human PF4 protein and its nucleotide coding sequence are known and provided herein. A polypeptide comprising the full-length PF4 protein or a mutant thereof including one or more point mutations thus can be chemically synthesized using conventional peptide synthesis or other protocols well-known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis in The Peptides: Analysis. Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an a-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin: hydroxymethyl resins: phenol resins, such as 4-(a-[2,4-dimethoxyphenyl]-Fmoc-aminomethyl) phenoxy resin: tert-alkyloxycarbonyl-hydrazidated resins, and the like. Such resins are commercially available and their methods of preparation are known by those of ordinary skill in the art. Briefly, the C-terminal N-α-protected amino acid is first attached to the solid support. The N-α-protecting group is then removed. The deprotected a-amino group is coupled to the activated a-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry. A Practical Textbook, 2nd Ed., Springer-Verlag (1993)).

### C. Recombinant Expression of Polypeptides

A PF4 protein of SEQ ID NO:1, its variant/mutant, or any fusion polypeptide comprising a wild-type PF4 protein or its variant/mutant can be produced using routine techniques in the field of recombinant genetics, relying on the polynucleotide sequences encoding the polypeptide disclosed herein.

To obtain high level expression of a nucleic acid encoding a desired polypeptide, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. One exemplary eukaryotic expression vector is an adenoviral vector, an adeno-associated vector, or a retroviral vector.

Standard transfection methods can be used to produce bacterial, mammalian, yeast, insect, or plant cell lines that express large quantities of a recombinant polypeptide (e.g., a PF4 dominant negative mutant), which is then purified using standard techniques (see. e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see. e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see. e.g., Sambrook and Russell, supra). It is only necessary that the particular genetic engineering procedure used be capable of successfully introducing at least one gene into the host cell capable of expressing the recombinant polypeptide.

When a recombinant polypeptide, e.g., a PF4 mutant, is expressed in host cells in satisfying quantity, its purification can follow the standard protein purification procedure including Solubility fractionation, size differential filtration, and column chromatography. These standard purification procedures are also suitable for purifying PF4 mutants or fusion polypeptides comprising a PF4 sequence (wild-type or mutant) obtained from chemical synthesis. The identity of the PF4 protein may be further verified by methods such as immunoassays (e.g., Western blot or ELISA) and mass spectrometry.

### III. Diagnosis and Treatment of Thrombocytopenia

Identification and diagnosis of conditions involving inappropriate blood clotting or undesirable inflammation, as well as methods of treating or reducing the risk of developing such conditions, are included in the present invention. As illustrated by the present inventors for the first time, the presence of anti-PF4 antibodies in a PF4-integrin complex plays a significant role in the pathogenesis of thrombocytopenia, including vaccine-induced thrombotic thrombocytopenia (VITT), heparin-induced thrombocytopenia (HIT), or autoimmune HIT (aHIT). Thus, a diagnostic method is developed based on the detection of anti-PF4 antibodies in the PF4-integrin complex (e.g., integrin αIIbβ3). Generally, the detection of anti-PF4 antibodies in such complex can be achieved by first isolating the PF4-integrin complex (e.g., integrin αIIbβ3) from a suitable sample, e.g., a blood sample, from a patient being tested due to having suspected clinical symptoms of thrombocytopenia and/or having experienced a potential triggering event, e.g., COVID or COVID vaccination, within a recent time frame (such as within 1, 2, 3, 4, 5 or up to 7 days) prior to the testing. An affinity-based isolation method is useful for isolation of the complex, for example, an agent with the ability to specifically bind the integrin molecule (e.g., integrin αIIbβ3) may be used as a “bait” to remove the PF4-integrin complex from a biological sample (e.g., a blood sample) taken from a patient being tested, an environment where many other biomolecules are present. Subsequently, an immunoassay such as ELISA may be employed to detect the presence of any anti-PF4 antibody in the complex.

Detecting the presence of anti-PF4 antibodies in the PF4-integrin complex isolated from a patient sample serves as a preliminary diagnostic indication of thrombocytopenia. At least one subsequent diagnostic method may be used to confirm the diagnosis of the condition, for example, conventional diagnostic methods of blood test (e.g., to determine the number of blood cells, especially platelet counts, in a blood sample) and physical examination (e.g., to observe signs of bleeding under the skin and examine abdomen for an enlarged spleen) may be employed to not only confirm the condition but also to aid devise an appropriate treatment plan.

Upon diagnosis of thrombocytopenia, a patient may receive treatment in accordance with the attending physician's determination of treatment plan, depending on the specific factors in the patient's medical and physical condition as well as the etiology, pathology, and severity of the condition. For instance, conventional treatment of administering a blood thinner may be employed. Further, blood or platelet transfusion may be performed, in the case of very low platelet count. Conventionally, three classes of medications are often administered for treating thrombocytopenia: antiaggregants (or antiplatelet drugs), anticoagulants, and thrombolytic agents. Other medications may be administered in cases where dysregulation of the immune system is deemed a contributing factor, for example, antihistamine and/or corticosteroid drugs may be used to suppress a hyper-inflammatory response by an inappropriately reactive immune system.

In addition to the use of medications, more invasive procedures may be utilized in rare cases of significant severity or urgency, including surgery to remove significant blood clots or to remove the spleen as well as to performing plasma exchange.

### V. Identification of Modulators of PF4-Integrin Association

### A. Molecular Docking

One method for rapidly assessing the potential of a test compound, e.g., a compound that has been proposed for use as a therapeutic agent in medical applications, to interfere with and inhibit PF4-integrin binding relies on the tool known as molecular docking.

A computer-based methodology, molecular docking was initially designed to predict the binding of small drug-like molecules to target proteins. As many diseases are caused by the malfunction of proteins and therapies are focused on the inhibition or activation of the target proteins, traditional lead generation methods for drug discovery normally entail assaying a large variety of interesting compounds against a specific protein known to be a disease target and hoping to observe a binding interaction. While more protein structures are determined experimentally using X-ray crystallography or nuclear magnetic resonance (NMR) spectroscopy, molecular docking is increasingly used as a tool in drug discovery. In the context of the present invention, molecular docking can be used to virtually screen new compounds in a similar way to experimental high-throughput screening as well as offering atomistic level insight to facilitate structure-based assessment of a binding relationship between the PF4 protein or integrin and a test compound as a rapid and effective means for preliminarily identifying compounds of possible therapeutic value, which, if preferred, may be further tested to confirm or eliminate the speculated binding characteristics.

The technology involved in molecular docking methodology has been in keen development for the past decades. A variety of software programs are now readily available for use in exploration of intermolecular interaction and are suitable either directly or upon modification in the practice of the present invention. Upon input of the specific chemical composition of a potential binding pair, such as the wild-type PF4 protein or integrin (e.g., integrin αIIbβ3) and a compound being analyzed for its potential capability to inhibit formation of the PF4-integrin complex, such software programs can predict a 3-dimensional binding orientation for the pair and generate a score corresponding to the predicted binding affinity. This score may then be utilized to assess the propensity of the two molecules, e.g., the test compound and the wild-type PF4 protein or integrin αIIbβ3, as a binding pair. For the purpose of the present invention, a pre-selected threshold value (e.g., a pre-determined value generated by the same software based on the binding between the PF4 protein or integrin αIIbβ3 and another compound known to bind the PF4 protein or integrin αIIbβ3) may be used to indicate a positive finding in the binding assessment (e.g., between the wild-type PF4 protein or integrin αIIbβ3 and another compound of unknown relevant binding profile). For a review of molecular docking software, see, e.g., Pagadala et al., Biophys Rev. 2017 April; 9 (2): 91-102.

### B. Physical Binding Assays

A second approach in assessing the potential effect of a compound focuses on the physical interaction between the compound and the wild-type PF4 protein or integrin αIIbβ3. As observed in this study, compounds that modulate the formation of the PF4-integrin complex may exert such effects by directly interacting with the wild-type PF4 protein, or with the integrin molecule. As such, an in vitro or cell-free screening method effective for providing a preliminary indication of whether a molecule is a modulator of PF4-integrin association relied on the detection of physical interaction or specific binding between a test compound and the PF4 protein (or integrin, especially integrin αIIbβ3).

Typically, a compound being screened for potential modulating effects on PF4-integrin binding is first placed together with the wild-type PF4 protein, or a pertinent integrin, under conditions generally allowing any potential binding between the compound and the wild-type PF4 protein (or integrin, e.g., especially integrin αIIbβ3) in an aqueous solution with appropriate salts and pH, the physical association between the test compound and the PF4 or integrin is then detected and quantitatively measured, for example, by determining a Kd value. If a decreased level of association is observed in comparison with the association level between the same PF4 or integrin protein and a “negative control” compound known to not specifically physically interact/bind with the PF4 or integrin protein, for example, by comparing the two Kd values, the test compound is preliminarily deemed a compound with likely capability to interfere with and inhibit the specific association between wild-type PF4 and integrin (e.g., integrin αIIbβ3). Conversely, if an increase in the level of association is observed compared to the “negative control” association level (e.g., by comparing the two Kd values), the test compound is preliminarily deemed as likely promoting PF4-integrin binding. Optionally, a compound that is preliminarily identified as a possible inhibitor of PF4-integrin association may be subject to further testing and investigation, for example, in cell-based assays or in experimental animal models for assessing its effect on thrombosis.

The initial screening step carried out in an in vitro or cell-free setting is suitable to be adapted in a high throughput system for simultaneously screening for a large number of test compounds for their potential binding to a potential partner. For example, an array of multiple test compounds having been immobilized to a solid substrate or support with each compound located at a distinct, pre-assigned, and thus individually identifiable location on the array may be contacted with the wild-type PF4 protein or integrin (e.g., integrin αIIbβ3) under conditions permissible for the compounds to bind to the PF4 or integrin protein. The presence of specific binding, defined as at least twice, preferably at least 5 times, 10, 20, 50, or 100 times over the background or “negative control” binding signal, between a test compound and the PF4 or integrin protein is then detected based on the presence of PF4 or integrin (e.g., as determined by an immunoassay) at an individually identifiable location on the array.

### VI. Pharmaceutical Compositions and Administration

The present invention also provides pharmaceutical compositions comprising an effective amount of a PF4 dominant negative mutant polypeptide for inhibiting a pro-inflammatory signal or a pro-thrombosis signal, therefore useful in both prophylactic and therapeutic applications designed for various diseases and conditions involving undesired inflammation and/or thrombosis. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249:1527-1533 (1990).

The pharmaceutical compositions of the present invention can be administered by various routes, e.g., oral, subcutaneous, transdermal, intramuscular, intravenous, or intraperitoneal. The routes of administering the pharmaceutical compositions include systemic or local delivery to a subject suffering from a condition exacerbated by inflammation at daily doses of about 0.01-5000 mg, preferably 5-500 mg, of a PF4 mutant polypeptide for a 70 kg adult human per day. The appropriate dose may be administered in a single daily dose or as divided doses presented at appropriate intervals, for example as two, three, four, or more subdoses per day.

For preparing pharmaceutical compositions containing a PF4 mutant polypeptide, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents: it can also be an encapsulating material.

In powders, the carrier is generally a finely divided solid that is in a mixture with the finely divided active component, e.g., a PF4 mutant polypeptide. In tablets, the active ingredient (the mutant polypeptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a PF4 mutant polypeptide with encapsulating material as a carrier providing a capsule in which the mutant (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a PF4 mutant polypeptide) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a PF4 mutant polypeptide) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing the active ingredient (e.g., a PF4 mutant) can be administered for prophylactic and/or therapeutic treatments. In therapeutic applications, compositions are administered to a patient already suffering from a condition that may be exacerbated by inappropriate blood clotting or an undesirable inflammatory reaction in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the mutant polypeptide per day for a 70 kg patient, with dosages of from about 5 mg to about 500 mg of the mutant polypeptide per day for a 70 kg patient being more commonly used.

In prophylactic applications, pharmaceutical compositions containing the active ingredient (e.g., a PF4 mutant) are administered to a patient susceptible to or otherwise at risk of developing a disease or condition involving inappropriate blood clotting or an undesirable inflammatory reaction in an amount sufficient to delay or prevent the onset of the symptoms. Such an amount is defined to be a “prophylactically effective dose.” In this use, the precise amounts of the inhibitor again depend on the patient's state of health and weight, but generally range from about 0.1 mg to about 2,000 mg of the mutant polypeptide for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound sufficient to effectively inhibit inappropriate blood clotting or an undesirable inflammatory reaction mediated by PF4 in the patient, either therapeutically or prophylactically.

### VII. Therapeutic Applications Using Nucleic Acids

A variety of inflammatory conditions or undesirable cell proliferation/angiogenesis can be treated by therapeutic approaches that involve introducing into a cell a nucleic acid encoding a PF4 dominant negative mutant polypeptide (e.g., R20E/R22E, K46E/R49E, or R20E/R22E/K46E/R49E) such that the expression of the mutant leads to reduced or abolished PF4-mediated cellular events in the cell. Those amenable to treatment by this approach include a broad spectrum of conditions involving inappropriate thrombosis and/or undesirable inflammation. For discussions on the application of gene therapy towards the treatment of genetic as well as acquired diseases, see, Miller Nature 357:455-460 (1992); and Mulligan Science 260:926-932 (1993).

### A. Vectors for Nucleic Acid Delivery

For delivery to a cell or organism, an inhibitory nucleic acid of the invention can be incorporated into a vector. Examples of vectors used for such purposes include expression plasmids capable of directing the expression of the PF4 mutants in the target cell. In other instances, the vector is a viral vector system wherein the polynucleotide is incorporated into a viral genome that is capable of transfecting the target cell. In a preferred embodiment, the inhibitory nucleic acid can be operably linked to expression and control sequences that can direct transcription of sequence in the desired target host cells. Thus, one can achieve reduced downstream effects medicated by PF4 under appropriate conditions in the target cell.

### B. Gene Delivery Systems

As used herein, “gene delivery system” refers to any means for the delivery of an inhibitory nucleic acid of the invention to a target cell. Viral vector systems useful in the introduction and expression of an inhibitory nucleic acid include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the inhibitory nucleic acid is inserted into such vectors to allow packaging of the gene construct, typically with accompanying viral DNA, followed by infection of a sensitive host cell and expression of the gene of interest.

Similarly, viral envelopes used for packaging gene constructs that include the inhibitory nucleic acid can be modified by the addition of receptor ligands or antibodies specific for a receptor to permit receptor-mediated endocytosis into specific cells (see. e.g., WO 93/20221, WO 93/14188, and WO 94/06923).

Retroviral vectors may also be useful for introducing the inhibitory nucleic acid of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins: the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra: Mulligan (1993), supra; and WO 92/07943.

The retroviral vector particles are prepared by recombinantly inserting the desired inhibitory nucleic acid sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, the inhibitory nucleic acid, thus eliminating or reducing unwanted inflammatory conditions.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA. 81:6349-6353 (1984); Danos and Mulligan Proceedings of the National Academy of Sciences, USA, 85:6460-6464 (1988); Eglitis et al. (1988), supra; and Miller (1990), supra.

### C. Pharmaceutical Formulations

When used for pharmaceutical purposes, the inhibitory nucleic acid is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can further include a stabilizer, an enhancer, and/or other pharmaceutically acceptable carriers or vehicles. A pharmaceutically acceptable carrier can contain a physiologically acceptable compound that acts, for example, to stabilize the inhibitory nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### D. Administration of Formulations

The formulations containing an inhibitory nucleic acid (e.g., encoding a PF4 dominant negative mutant) can be delivered to any tissue or organ using any delivery method known to the ordinarily skilled artisan. In some embodiments of the invention, the nucleic acid is formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

The formulations containing the inhibitory nucleic acid are typically administered to a cell. The cell can be provided as part of a tissue or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the inhibitory nucleic acid is introduced into cells by such methods as microinjection, calcium phosphate precipitation, liposome fusion, ultrasound, electroporation, or biolistics. In further embodiments, the nucleic acid is taken up directly by the tissue of interest.

In some embodiments of the invention, the inhibitory nucleic acid is administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223 (2):116-26 (1996); Dalesandro et al., J. Thorac. Cardi. Surg., 11(2):416-22 (1996); and Makarov et al., Proc. Natl. Acad. Sci. USA 93 (1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg inhibitory nucleic acid per patient are typical. Doses generally range between about 0.01 and about 50 mg per kilogram of body weight, preferably between about 0.1 and about 5 mg/kg of body weight or about 10⁸-10¹⁰ or 10¹² viral particles per injection. In general, the dose equivalent of a naked nucleic acid from a vector is from about 1 μg-100 μg for a typical 70 kg patient, and doses of vectors which include a retroviral particle are calculated to yield an equivalent amount of an inhibitory nucleic acid.

### VIII. Kits

The invention also provides kits for preventing or treating thrombocytopenia or treating a condition involving undesirable inflammatory responses including autoimmune responses by inhibiting the specific binding between PF4 and integrin according to the method of the present invention. The kits typically include a first container that contains a pharmaceutical composition having an effective amount of an inhibitor of PF4-integrin binding, such as a PF4 dominant negative mutant or an anti-PF4 nanobody that reduces or abolishes PF4-integrin binding, optionally with a second container containing an anti-thrombotic agent, which may belong to any of the following 3 classes of drugs: (A) antiplatelet drugs (also known as antiaggregants), including irreversible cyclooxygenase inhibitors (e.g., aspirin and triflusal), adenosine diphosphate (ADP) receptor inhibitors (e.g., cangrelor, clopidogrel, prasugrel, ticagrelor, and ticlopidine), phosphodiesterase inhibitors (e.g., cilostazol), protease-activated receptor-1 (PAR-1) antagonists (e.g., vorapaxar), glycoprotein IIB/IIIA inhibitors (e.g., abciximab, eptifibatide, and tirofiban), adenosine reuptake inhibitors (e.g., dipyridamole), and thromboxane inhibitors (e.g., terutroban); (B) anticoagulants, including coumarins (vitamin K antagonists), heparin and derivative substances (e.g., low molecular weight heparin or LMWH), and synthetic pentasaccharide inhibitors of factor Xa (e.g., fondaparinux, idraparinux, and idrabiotaparinux); and (C) thrombolytic agents, which are primarily recombinantly produced fibrinolytics proteins, e.g., streptokinase (Kabikinase), urokinase, recombinant tissue plasminogen activators (rtPA), alteplase (Activase or Actilyse), reteplase (Retavase), Tenecteplase, and anistreplase (Eminase).

In some cases, the kits will also include informational material containing instructions on how to dispense the pharmaceutical composition, including description of the type of patients who may be treated (e.g., a person suffering from abnormal blood clotting or at risk of developing thrombocytopenia, including due to COVID or COVID vaccination), the schedule (e.g., dose and frequency of administration) and route of administration, and the like.

### Examples

The following examples are provided by way of illustration only and not by way of limitation. Those of skill in the art will readily recognize a variety of non-critical parameters that could be changed or modified to yield essentially the same or similar results.

### Introduction

It has been reported that vaccination for SARS-CoV-2 may result in a vaccine-induced catastrophic thrombotic thrombocytopenia (VITT) disorder. This disorder presents as extensive thrombosis in atypical sites, primarily in the cerebral venous, alongside thrombocytopenia and the production of autoantibody against platelet-factor 4 (PF4, chemokine CXCL4). This rare adverse effect extremely resembles the clinical presentation of the classical immune-mediated heparin-induced thrombocytopenia (HIT) disorder, which is induced by anti-PF4/heparin complex and occurs following exposure to heparin (1-8). VITT is also very similar to autoimmune HIT (aHIT), which is induced by anti-PF4 but none of these patients had been pre-exposed to heparin before disease onset. Anti-PF4 autoantibodies have also been detected in several autoimmune diseases (e.g., SLE, Systemic sclerosis, and RA) (9-11). PF4 is one of the most abundant proteins in platelet granules and PF4 is present at >1 μg/ml concentrations in plasma. It is unclear how anti-PF4 induces thrombocytopenia. A current model of thrombotic thrombocytopenia suggests that (1) anti-PF4 binds to PF4 and induces PF4 clustering, (2) the complex binds to platelets by binding to the FcRγIIA receptor and proteoglycans of platelets, leading to (3) platelet activation and aggregation (8). Although activation of αIIbβ3 is known to be a key event in platelet aggregation and thrombus formation, integrins are not involved in this model.

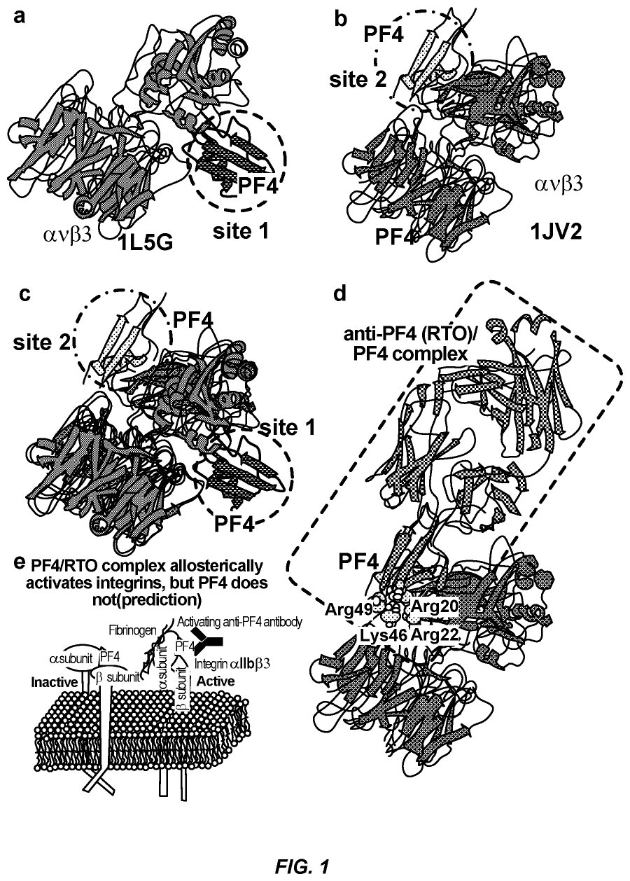

Integrins are a superfamily of αβ heterodimers that were originally identified as receptors for extracellular matrix proteins (12). PF4 is known to bind to αvβ3 (13) and Mac-1 (14). We previously discovered that the chemokine domain of pro-inflammatory chemokine CX3CL1 is a ligand for integrins αvβ3 and αvβ1 and bound to the classical ligand-binding site of integrins (site 1) (15). We showed that CX3CL1 activated soluble integrin αvβ3 in cell-free conditions in ELISA-type activation assay (16). In this assay, we coated the wells of 96-well plate with specific integrin ligands and incubated with soluble integrins in the presence of CX3CL1 in 1 mM Ca²⁺ to keep integrin inactive. We detected the increase in integrin binding to immobilized ligand γC399tr, a fibrinogen fragment, indicating that soluble integrins were activated by CX3CL1. We predicted that CX3CL1 binds to the second-binding site in integrin headpiece, allosteric site (site 2), which is distinct from the classical ligand-binding site (site 1), by docking simulation of the interaction between the closed/inactive integrin αvβ3 (1JV2.pdb) and CX3CL1 (16). Site 2 is located on the opposite side of site 1 in the integrin headpiece (FIG. 1). We also found that another pro-inflammatory chemokines SDF-1 (CXCL12) activates integrins αvβ3, α4β1, and α5β1 by binding to site 2 (17). 25-Hydroxycholesterol, a mediator of inflammatory signals in innate immunity, is known to bind to integrin site 2 and induce integrin activation and inflammatory signaling, leading to over-production of inflammatory cytokines in monocytes (18). It has thus been proposed that site 2 plays a critical role in inflammation. We thus hypothesized that PF4 binds to site 2 and anti-PF4 modifies this interaction.

In the present study, we showed that PF4 bind to site 1. PF4 is predicted to bind to site 2, but did not activate integrins by itself. Surprisingly, anti-PF4/PF4 complex potently activated integrins. We generated PF4 mutants defective in site 2 binding by introducing mutations in the predicted site 2 binding interface of PF4. A PF4 mutant/anti-PF4 complex was defective in activating integrins αIIbβ3 and αvβ3. Furthermore, this PF4 mutant acted as an antagonist of wild-type PF4/anti-PF4-induced integrin activation. These findings suggest that anti-PF4/PF4 requires to bind to site 2 for activating integrins. We propose that anti-PF4/PF4 complex induces integrin αIIbβ3 activation, and induce subsequent αIIbβ3-fibrinogen bridge, leading to platelet aggregation. Also, anti-PF4 may be involved in the pathogenesis of autoimmune diseases by allosterically activating αvβ3 and other integrins in non-platelet cells (e.g., monocytes).

### Results

PF4 Specifically Binds to Site 1 and Site 2 of Integrin αvβ3

Although PF4 is known to bind to integrins αvβ3, the specifics of interaction are unclear. To predict how PF4 binds to integrin, we performed docking simulation of interaction between PF4 (1RHP.pdb) and integrin αvβ3 using autodock3. The 3D structure of αvβ3 was used since active and inactive conformers are well defined. In our docking studies, PF4 is predicted to bind to site 1 (docking energy −24.3 kcal/mol) of active conformer of integrin αvβ3 (open headpiece/active, 1L5G.pdb) (FIG. 1a).

Docking simulation of interaction between PF4 (1RHP.pdb) and inactive conformer of integrin αvβ3 (closed headpiece/inactive 1JV2.pdb) predicted that PF4 binds to site 2 (docking energy 21 kcal/mol) (FIG. 1b), predicting that PF4 allosterically activate integrins as in CX3CL1 or CXCL12.

When FIGS. 1a and 1b are superposed, it is clear that PF4 binds to two distinct binding site (site 1 and site 2) (FIG. 1c).

Table 1 shows amino acid residues in PF4 that are involved in site 2 binding. We superposed the PF4/RTO complex (1RHP.pdb) and PF4/αvβ3 complex (FIG. 1d). It is predicted that RTO, PF4 and integrin can co-exist without steric hindrance. Also, the predicted site 2-binding site in PF4 is outside of PF4 tetramer, and PF4 tetramer is predicted to bind to site 2 without steric hindrance (not shown). Positions of amino acids in PF4 that are involved in site 2 binding is shown in Table 1.

The docking model studies predicts a model, in which PF4 binding to integrin site 2 does not activate integrins but anti-PF4/PF4 complex binds to site 2 and activates integrins (FIG. 1e).

PF4 Binds to Site 1 of Activated Soluble Integrins αIIbβ3 and αvβ3.

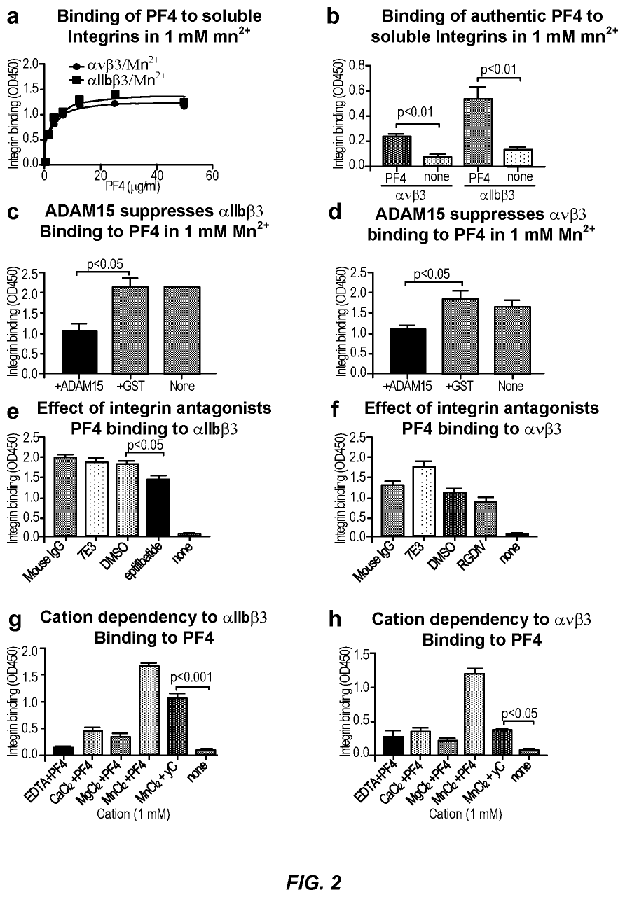

Consistent with the prediction, we showed that PF4 bound to soluble αIIbβ3 and αvβ3, which are activated by 1 mM Mn²⁺ in ELISA type binding assays in cell-free conditions (FIG. 2a).

To confirm that the binding of PF4 to soluble integrins is due to abnormality of our PF4 preparations, we tested if authentic PF4 (Invitrogen) behaves similarly to PF4 that was generated in our lab. We obtained similar results using authentic PF4 (FIG. 2b) indicating that the binding of PF4 to integrins is a property of PF4. All experiments were performed using PF4 preparations generated in our lab if not indicated otherwise.

We found that the disintegrin domain of ADAM15, which is known to bind to integrins αvβ3 (21) and αIIbβ3 (22), inhibited the binding of PF4 to integrins, but control GST did not (FIGS. 2c and 2d). This indicates that the PF4 binding to these integrins is specific.

However, known antagonists for these integrins effectively inhibited PF4 binding to these integrins, except for weak inhibition by eptifibatide (specific to αIIbβ3) weakly inhibited (FIGS. 2e and 2f). Mn²⁺ (1 mM) supported PF4 binding to site 1 well but other cations did not. Also, the full-length fibrinogen C-terminal domain (γC151-411), a ligand for αIIbβ3 and αvβ3, bound to these integrins as a positive control (FIGS. 2g and 2h).

### PF4/Anti-PF4 (RTO) Complex Potently Activates Integrins at Physiological PF4 Concentrations, Although PF4 Itself Did not.

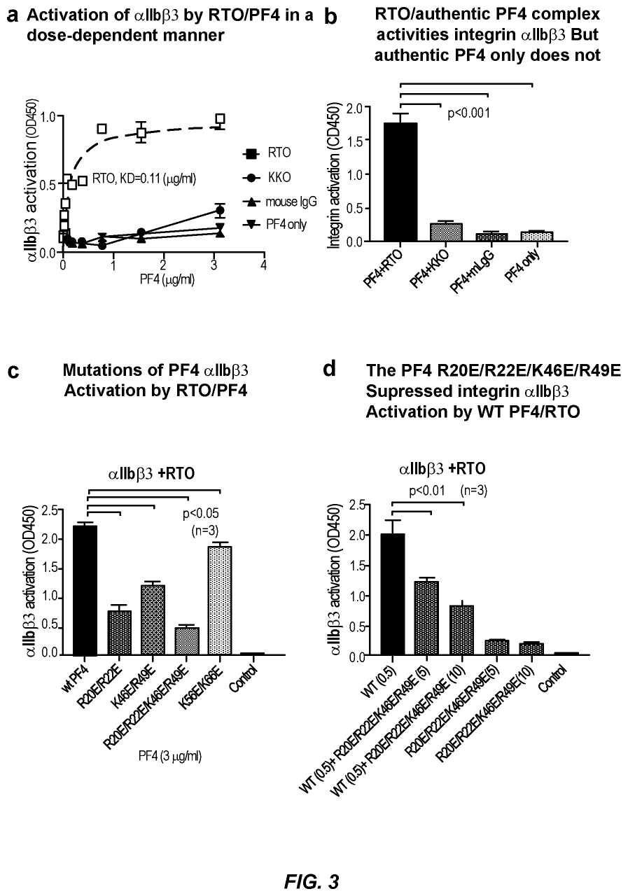

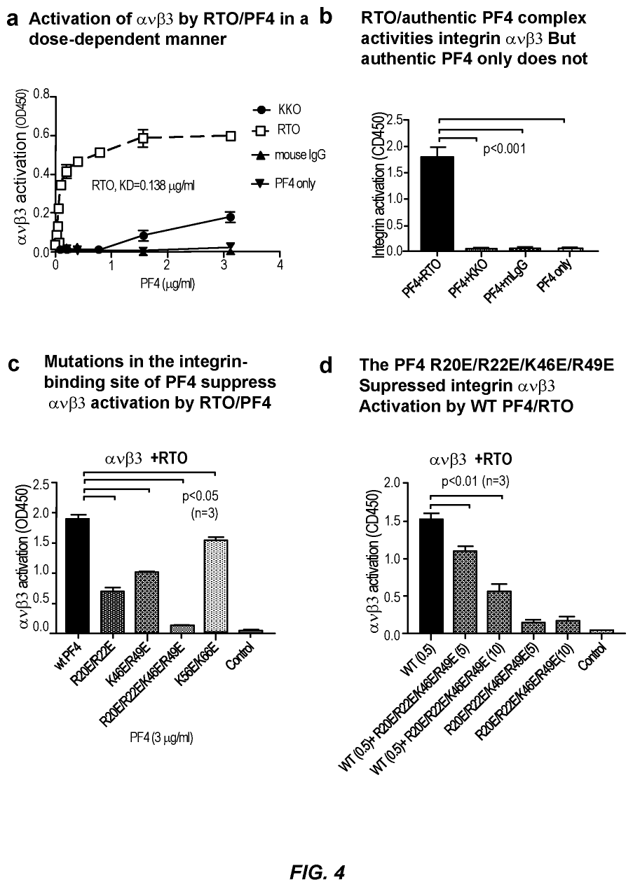

Previous studies showed that inflammatory chemokines CX3CL1, CXCL12, and CCL5 bound to site 2 and activated integrins αIIbβ3 and αvβ3 (18, 19). Although PF4 is predicted to bind to site 2, PF4 by itself did not activate αIIbβ3 or αvβ3 (FIGS. 3a and 4a).

A murine mAb KKO to human (h) PF4/heparin complexes is known to bind specifically to hPF4/heparin complexes and induces HIT (pathogenic mAb) (23). Murine anti-hPF4 mAb RTO does not require heparin for binding to PF4 and does not induce HIT (non-pathogenic) (23). We studied if RTO and KKO influence the ability of PF4 to induce integrin activation. We expected that pathogenic KKO will activate αIIbβ3 and αvβ3 by binding to PF4 and non-pathogenic RTO will not. Unexpectedly, the RTO (at 10 μg/ml)/PF4 complex markedly activated integrins but KKO/PF4 complex did not in a heparin-independent manner in ELISA-type activation assays in 1 mM Ca²⁺. KKO/PF4 complex did not activate integrins (FIGS. 3a and 4a). We also found that RTO/PF4 complex activated αIIbβ3 and αvβ3 at physiological concentrations of PF4 (<1 μg/ml), but PF4 itself did not (FIGS. 3a and 4a).

This is the first clue that anti-PF4/PF4 complex is potentially involved in integrin αIIbβ3 activation, a trigger of platelet aggregation. Since heparin-independent anti-PF4 has been detected in autoimmune diseases and the levels of anti-PF4 correlate with disease progression (11, 13), activation of vascular αvβ3 by anti-PF4/PF4 complex may be involved in the pathogenesis of autoimmune diseases. RTO can be used as a model of heparin-independent anti-PF4 detected in VITT, aHIT, and autoimmune diseases.

### PF4 Mutant Defective in Site 2 Binding is Defective in Integrin Activation and Acted as an Antagonist for PF4/RTO-Induced Integrin Activation.

The predicted RTO-binding site and site 2-binding site in PF4 are distinct. To determine if PF4/RTO complex activates integrins by binding to site 2, we developed PF4 mutants that are defective in site 2 binding by introducing mutations in the site 2-binding interface in PF4 predicted by docking simulation (FIG. 1). Arg20, Arg22, Lys46, Arg49, Lys65, and Lys66 in the integrin-binding interface of PF4 were mutated to Glu. Positions of these amino acid residues are shown in FIG. 1d. The R20E/R22E and K46E/R49E mutations showed reduced ability to mediate RTO/PF4-induced integrin activation (FIGS. 3c and 4c). The combined PF4 mutant (R20E/R22E/K46E/R49E) most effectively reduced RTO/PF4-induced integrin activation (FIGS. 3c and 4c). Notably, this mutant suppressed integrin activation induced by PF4/RTO complex in a dose-dependent manner (dominant-negative effect) (FIGS. 3d and 4d). This PF4 mutant binds to anti-PF4 (heparin-independent) but cannot induce integrin activation since it cannot bind to site 2. The PF4 mutant competes with wild-type PF4 complex for binding to anti-PF4.

### Discussions

The present study establishes that RTO/PF4 complex potently activated integrins by binding to site 2, although PF4 by itself did not activate integrins at physiological concentrations of PF4. Since the RTO/PF4 mutant defective in binding to site 2 did not activate integrins and acted as an antagonist of anti-PF4/PF4-induced integrin activation, PF4 binding to site 2 is critical for RTO/PF4-induced integrin activation. It is likely that RTO binding to PF4 changed the phenotype of PF4 and activated integrins upon binding to site 2. This phenomenon mimics the anti-PF4-induced aHIT and VITT, and potentially connects thrombocytopenia, platelet integrin αIIbβ3, and anti-PF4. It is interesting that KKO (heparin-dependent anti-human PF4 induces HIT, but RTO (heparin-independent anti-human PF4) does not (4). One possibility is that heparin-independent and dependent TT are induced by totally different mechanisms. Therefore, RTO is a model of heparin-independent autoantibodies to PF4 that induce thrombocytopenia. Our studies indicate that anti-PF4/PF4-induced αIIbβ3 activation causes thrombocytopenia. It is believed that anti-PF4 like RTO can change the phenotype of PF4 and anti-PF4/PF4 complex can activate integrins by binding to site 2. The PF4 mutant defective in site 2 binding may suppress thrombosis by blocking anti-PF4-induced integrin αIIbβ3 activation and has potential as an antagonist for allosteric integrin activation. The ELISA-type activation of integrins by anti-PF4/PF4 complex can be potentially useful to detect heparin-independent anti-PF4 in patients' blood.

Also, RTO/PF4 complex can activate vascular integrin αvβ3 in an allosteric manner. Since anti-PF4 is detected in several autoimmune diseases as well, and PF4 does not activate αvβ3 (and perhaps other integrins), anti-PF4 stimulate integrin activation in cell types (e.g., monocytes) other than platelets. The levels of heparin-independent anti-PF4 is known to correlate with disease activity index in SLE patients (11). Therefore, the same PF4 mutant is believed to block vascular inflammation induced by anti-PF4 in autoimmune diseases.

TABLE 1
Amino acid residues involved in PF4-integrin αvβ3 interaction
predicted by docking simulation

| PF4 (1RHP.pdb) | αv (1JV2.pdb) | β3 (1JV2.pdb) |
|---|---|---|
| Thr15, Thr16, Ser17, | Glu15, Gly16, Ser17, | Pro160, Met165, Lys235, |
| Gln18, Val19, Arg20, | Tyr18, Phe19, Lys42, | Gly264, Ile265, Val266, |
| Pro21, Arg22, His23, | Asn44, Ile50, Val51, | Gln267, Asp270, Gln272, |
| Thr25, Lys46, Asn47, | Glu52, Trp93, Ala396, | Cys273, His274, Val275, |
| Arg49, Asp54, Leu55, | Ala397, Arg398, Ser399, | Gly276, Ser277, Asp278, |
| Gln56, Ala57, Pro58, | Met400, Phe427, | His280, Tyr281, Ser282, |
| Leu59, Lys62, Lys65, | Gly428, Val429, Asp430 | Ala283, Thr285, Thr286, |
| Lys66, Leu67, Glu69, | | Met287 |
| Ser70 | | |

Amino acid residues within 0.6 nm between PF4 and αvβ3 were selected using pdb viewer (version 4.1). Amino acid residues in PF4 selected for mutagenesis are shown in bold.

### Materials and Methods

### Fibrinogen γ-Chain C-Terminal Residues 390-411, a Specific Ligand for αIIbβ3 Fused to GST

cDNA encoding (6 His tag and Fibrinogen y-chain C-terminal residues 390-411) [HHHHHH]NRLTIGEGQQHHLGGAKQAGDV (SEQ ID NO:2) was conjugated with the C-terminus of GST (designated γC390-411) in pGEXT2 vector (BamHI/EcoRI site). The protein was synthesized in E. coli BL21 and purified using glutathione affinity chromatography.

The fibrinogen γ-chain C-terminal domain (γC399tr, residues 151-399), a specific ligand for αvβ3 has been previously described (22). The disintegrin domain of ADAM15 fused to GST (ADMA 15 disintegrin) and parent GST were synthesized as previously described (19).

### PF4

The cDNA encoding PF4 was synthesized and subcloned into the BamHI/EcoRI site of pET28a vector. Protein synthesis was induced by IPTG in E. coli BL21 and protein was synthesized as insoluble inclusion bodies and purified in Ni-NTA affinity chromatography under denaturing conditions and renatured as described (17). PF4 used as an authentic control, and anti-PF4 antibodies RTO and KKO were obtained from Invitrogen.

### ELISA-Type Integrin Activation Assays (17)

Wells of 96-well microtiter plates were coated with γC390-411 (a specific ligand for αIIbβ3) or γC399tr (a specific ligand for αvβ3). Remaining protein binding sites were blocked with BSA. Soluble recombinant αIIbβ3 or αvβ3 (AgroBio, 1 μg/ml) was pre-incubated with chemokines PF4 or anti-PF4/PF4 for 10 min at room temperature and was added to the wells and incubated in HEPES-Tyrodes buffer with 1 mM CaCl2 for 1 h at room temperature. After unbound αIIbβ3 or αvβ3 was removed by rinsing the wells with binding buffer, bound αIIbβ3 or αvβ3 was measured using anti-integrin B3 mAb (AV-10) followed by HRP-conjugated goat anti-mouse IgG and peroxidase substrates.

### ELISA-Type Integrin Binding Assays (15)

Wells of 96-well microtiter plates were coated with PF4. Remaining protein binding sites were blocked by incubating with BSA. After washing with PBS, soluble recombinant αIIbβ3 or αvβ3 (1 μg/ml) was added to the wells and incubated in HEPES-Tyrodes buffer with 1 mM MnCl₂ for 1 h at room temperature. Bound αIIbβ3 or αvβ3 was measured using anti-integrin β3 mAb (AV-10) followed by HRP-conjugated goat anti-mouse IgG and peroxidase substrates.

### Docking Simulation

Docking simulation of interaction between PF4 and integrin αvβ3 (open headpiece form 1L5G, or closed headpiece form, PDB code 1JV2) was performed using AutoDock3 as described previously (24). We used the headpiece (residues 1-438 of αv and residues 55-432 of B3) of αvβ3 (closed form, 1JV2.pdb). Cations were not present in integrins during docking simulation, as in the previous studies using αvβ3 (open headpiece form, 1L5G.pdb) (24, 25). The ligand is presently compiled to a maximum size of 1024 atoms. Atomic solvation parameters and fractional volumes were assigned to the protein atoms by using the AddSol utility, and grid maps were calculated by using AutoGrid utility in AutoDock 3.05. A grid map with 127×127×127 points and a grid point spacing of 0.603 Å included the headpiece of αvβ3. Kollman ‘united-atom’ charges were used. AutoDock 3.05 uses a Lamarckian genetic algorithm (LGA) that couples a typical Darwinian genetic algorithm for global searching with the Solis and Wets algorithm for local searching. The LGA parameters were defined as follows: the initial population of random individuals had a size of 50 individuals; each docking was terminated with a maximum number of 1×10⁶ energy evaluations or a maximum number of 27 000 generations, whichever came first; mutation and crossover rates were set at 0.02 and 0.80, respectively. An elitism value of 1 was applied, which ensured that the top-ranked individual in the population always survived into the next generation. A maximum of 300 iterations per local search were used. The probability of performing a local search on an individual was 0.06, whereas the maximum number of consecutive successes or failures before doubling or halving the search step size was 4.

### Other Methods

Treatment differences were tested using ANOVA and a Tukey multiple comparison test to control the global type I error using Prism 7 (Graphpad Software).

All patents, patent applications, and other publications cited in this application, including published amino acid or polynucleotide sequences identified by GenBank Accession Numbers and the like, are incorporated by reference in the entirety for all purposes.

### REFERENCES

1. Mori S, Wu C Y, Yamaji S, Saegusa J. Shi B, Ma Z, et al. Direct binding of integrin alphavbcta3 to FGF1 plays a role in FGF1 signaling. J Biol Chem. 2008; 283(26):18066-75.
2. Saegusa J, Akakura N, Wu C Y, Hoogland C, Ma Z, Lam K. S, et al. Pro-inflammatory secretory phospholipase A2 type IIA binds to integrins alphavbeta3 and alpha4beta1 and induces proliferation of monocytic cells in an integrin-dependent manner. J Biol Chem. 2008; 283(38):26107-15.
3. Brodard J, Kremer Hovinga J A, Fontana P, Studt J D, Gruel Y, Greinacher A, COVID-19 patients often show high-titer non-platelet-activating anti-PF4/heparin IgG antibodies. J Thromb Haemost. 2021.
4. Datta P, Zhang F, Dordick J S, Linhardt R J, Platelet factor 4 polyanion immune complexes: heparin induced thrombocytopenia and vaccine-induced immune thrombotic thrombocytopenia. Thromb J. 2021; 19(1):66.
5. Dickhout A, Tullemans B M E, Heemskerk J W M, Thijssen V, Kuijpers M J E, Koenen R R. Galectin-1 and platelet factor 4 (CXCL4) induce complementary platelet responses in vitro. PLOS One. 2021; 16(1):e0244736.
6. Favaloro E J, Mohammed S, Plasma vs serum as test sample for the chemiluminescent AcuStar HemosIL HIT-IgG (PF4-H) assay. Int J Lab Hematol. 2021; 43(1):e41-c4.
7. Favaloro E J, Mohammed S, Donikian D, Kondo M, Hamdam S, Hocker N, et al. Effect of sample heat inactivation on test levels of HIT-IgG (PF4-H) detected by the ACL AcuStar. Thromb Res. 2021; 200:12-5.
8. Marashi-Sabouni Z, Vayne C, Ibrahim-Kosta M, Guidon C, Loundou A, Guery E A, et al. Clinical validation of immunoassay HemosIL® AcuStar HIT-IgG (PF4-H) in the diagnosis of Heparin-induced thrombocytopenia. J Thromb Thrombolysis. 2021; 52(2):601-9.
9. Samuelson Bannow B, Warad D M, Jones C G, Pechauer S M, Curtis B R, Bougie D W, et al. A prospective, blinded study of a PF4-dependent assay for HIT diagnosis. Blood. 2021; 137(8):1082-9.
10. Vayne C, Nguyen T H, Rollin J, Charuel N, Poupon A, Pouplard C, et al. Characterization of New Monoclonal PF4-Specific Antibodies as Useful Tools for Studies on Typical and Autoimmune Heparin-Induced Thrombocytopenia. Thromb Haemost. 2021; 121(3):322-31.
11. Satoh T, Tanaka Y, Okazaki Y, Kaburaki J, Ikeda Y, Kuwana M, Heparin-dependent and -independent anti-platelet factor 4 autoantibodies in patients with systemic lupus erythematosus. Rheumatology (Oxford). 2012; 51(9):1721-8.
12. Lande R, Mennella A, Palazzo R, Pietraforte I, Stefanantoni K, Iannace N, et al. Anti-CXCL4 Antibody Reactivity Is Present in Systemic Sclerosis (SSc) and Correlates with the SSc Type I Interferon Signature. Int J Mol Sci. 2020; 21(14).
13. Izumi M, Sakai T, Shirakawa A, Kozuru H, Jiuchi Y, Izumi Y, et al. Reduced induction of anti-PF4/heparin antibody in RA patients after total knee arthroplasty. Arthritis Res Ther. 2016; 18:191.
14. Takada Y, Ye X, Simon S. The integrins. Genome Biol. 2007; 8(5):215.
15. Aidoudi S, Bujakowska K, Kieffer N, Bikfalvi A. The CXC-chemokine CXCL4 interacts with integrins implicated in angiogenesis. PLOS One. 2008; 3(7):e2657.
16. Lishko V K, Yakubenko V P, Ugarova T P, Podolnikova N P. Leukocyte integrin Mac-1 (CD11b/CD18, alphaMbeta2. CR3) acts as a functional receptor for platelet factor 4. J Biol Chem. 2018; 293(18):6869-82.
17. Fujita M, Takada Y K, Takada Y. Integrins alphavbeta3 and alpha4beta1 act as coreceptors for fractalkine, and the integrin-binding defective mutant of fractalkine is an antagonist of CX3CR1. J Immunol. 2012; 189(12):5809-19.
18. Fujita M, Takada Y K, Takada Y. The chemokine fractalkine can activate integrins without CX3CR1 through direct binding to a ligand-binding site distinct from the classical RGD-binding site. PLOS One. 2014; 9(5):e96372.
19. Fujita M, Davari P, Takada Y K, Takada Y. Stromal cell-derived factor-1 (CXCL12) activates integrins by direct binding to an allosteric ligand-binding site (site 2) of integrins without CXCR4. Biochem J. 2018; 475(4):723-32.
20. Pokharel S M, Shil N K, Gc J B, Colburn Z T, Tsai S Y, Segovia J A, et al. Integrin activation by the lipid molecule 25-hydroxycholesterol induces a proinflammatory response. Nat Commun. 2019; 10(1):1482.
21. Zhang X P, Kamata T, Yokoyama K, Puzon-McLaughlin W, Takada Y. Specific interaction of the recombinant disintegrin-like domain of MDC-15 (metargidin, ADAM-15) with integrin alphavbeta3. J Biol Chem. 1998; 273(13):7345-50.
22. Langer H, May A E, Bultmann A, Gawaz M. ADAM 15 is an adhesion receptor for platelet GPIIb-IIIa and induces platelet activation. Thromb Haemost. 2005; 94(3):555-61.
23. Arepally G M, Kamei S, Park K S, Kamei K, Li Z Q, Liu W, et al. Characterization of a murine monoclonal antibody that mimics heparin-induced thrombocytopenia antibodies. Blood. 2000; 95(5):1533-40.
24. Yokoyama K, Zhang X P, Medved L, Takada Y. Specific binding of integrin alpha v beta 3 to the fibrinogen gamma and alpha E chain C-terminal domains. Biochemistry. 1999; 38(18):5872-7.

1. A platelet factor 4 (PF4)-derived polypeptide comprising the amino acid sequence of SEQ ID NO:1 with one or more mutations at residues 20, 22, 46, 49, 65, and 66, preferably one or more Glu substitutions at residues 20, 22, 46, 49, 65, and 66, preferably at residues 20, 22, 46, and 49, and having decreased binding to integrin.

2. The PF4-derived polypeptide of claim 1, comprising the amino acid sequence of SEQ ID NO: 1 with Glu substitutions at residues 20 and 22 and/or at residues 46 and 49.

3. The PF4-derived polypeptide of claim 2, comprising the amino acid sequence of SEQ ID NO:1 with Glu substitutions at residues 20, 22, 46, and 49.

4. The PF4-derived polypeptide of claim 1, wherein the integrin is integrin αIIbβ3 or integrin αvβ3.

5. A method for diagnosing thrombocytopenia in a patient, comprising detecting in a blood sample taken from the patient presence of anti-PF4 antibody in a PF4-integrin complex.

6. The method of claim 5, wherein the thrombocytopenia is a vaccine-induced thrombotic thrombocytopenia (VITT), heparin-induced thrombocytopenia (HIT), autoimmune HIT (aHIT).

7. The method of claim 5, wherein the thrombocytopenia is VITT, and wherein the patient has received COVID-19 vaccination within the past 24-48 hours.

8. The method of claim 5, wherein the thrombocytopenia is aHIT, and wherein the patient has been diagnosed with COVID-19.

9. The method of claim 5, wherein the detecting step comprises an ELISA assay for anti-PF4 antibody.

10. The method of claim 5, wherein the integrin is integrin αIIbβ3 or integrin αvβ3.

11. A method for preventing or treating thrombocytopenia in a patient in need thereof, comprising administering to the patient an effective amount of an inhibitor of binding between PF4 and integrin.

12. The method of claim 11, wherein the thrombocytopenia is a vaccine-induced thrombotic thrombocytopenia (VITT), heparin-induced thrombocytopenia (HIT), autoimmune HIT (aHIT).

13. The method of claim 11, wherein the thrombocytopenia is VITT, and wherein the patient has received COVID-19 vaccination within the past 24-48 hours.

14. The method of claim 11, wherein the thrombocytopenia is aHIT, and wherein the patient has been diagnosed with COVID-19.

15. The method of claim 11, wherein the inhibitor is a compound that interferes with binding between PF4 and integrin.

16. The method of claim 15, wherein the inhibitor is an antibody against PF4 that interferes with binding between PF4 and integrin.

17. The method of claim 16, wherein the antibody is a single chain antibody (ScFv) or a nanobody for PF4.

18. The method of claim 15, wherein the inhibitor is a PF4 mutant that does not bind integrin.

19. The method of claim 18, wherein the PF4 mutant comprises one or more mutations within a region of PF4 interacting with integrin.

20. The method of claim 18, wherein the PF4 mutant comprises the amino acid sequence of SEQ ID NO:1 with one or more mutations at residues 20, 22, 46, 49, 65, and 66, preferably one or more Glu substitutions at residues 20, 22, 46, 49, 65, and 66, preferably residues 20, 22, 46, and 49.

21. The method of claim 20, wherein the PF4 mutant comprises the amino acid sequence of SEQ ID NO: 1 with Glu substitutions at residues 20 and 22 and/or at residues 46 and 49.

22. The method of claim 21, wherein the PF4 mutant comprises the amino acid sequence of SEQ ID NO:1 with Glu substitutions at residues 20, 22, 46, and 49.

23. The method of claim 11, wherein the integrin is integrin αIIbβ3 or integrin αvβ3.

24. A method for identifying an inhibitor of PF4-integrin binding, comprising the steps of
(1) contacting an integrin and a polypeptide comprising the PF4 amino acid sequence, in the presence of a test compound, under conditions permissible for PF4-integrin binding; and
(2) detecting the level of polypeptide-integrin binding, wherein a decrease in the level of binding when compared with the level of binding in the absence of the test compound indicates the compound as an inhibitor of PF4-integrin binding.

25. The method of claim 21, wherein the integrin is expressed on a cell surface.

26. The method of claim 21, wherein step (1) is performed in a cell-free system.

27. The method of any one of claim 24, wherein the integrin is integrin αIIbβ3 or integrin αvβ3.

28. A kit for inhibiting thrombosis, comprising a first container containing an inhibitor of binding between PF4 and integrin and a second container containing an anti-thrombosis therapeutic agent.

29. The kit of claim 28, wherein the inhibitor is a PF4 mutant comprising the amino acid sequence of SEQ ID NO:1 with one or more mutations at residues 20, 22, 46, 49, 65, and 66, preferably one or more Glu substitutions at residues 20, 22, 46, 49, 65, and 66, preferably at residues 20, 22, 46, and 49.

30. The kit of claim 28, wherein the PF4 mutant comprises the amino acid sequence of SEQ ID NO: 1 with Glu substitutions at residues 20 and 22 and/or at residues 46 and 49.

31. The kit of claim 30, wherein the PF4 mutant comprises the amino acid sequence of SEQ ID NO: 1 with Glu substitutions at residues 20, 22, 46, and 49.

32. The kit of claim 28, wherein the integrin is integrin αIIbβ3 or integrin αvβ3.
